(19) United States
(12) Patent Application Publication
(10) Pub. No.: US 2006/0111388 A1
(43) Pub. Date: May 25, 2006

(54) Phenanthroline and derivatives thereof used to lower intraocular pressure in an affected eye

(75) Inventors: Adnan Dibas, Arlington, TX (US); Thomas Yorio, Burleson, TX (US); Ganesh Prasanna, San Diego, CA (US)

Correspondence Address: T. Ling Chwang, Suite 600, 2435 N. Central Expressway, Richardson, TX, 75080, US

(73) Assignee: University of North Texas Health Science Center at Fort Worth, Fort Worth, TX (US)

(21) Appl. No.: 11/282,277

(22) Filed: Nov. 18, 2005

Related U.S. Application Data
(60) Provisional application No. 60/629,786, filed on Nov. 19, 2004.

(51) Int. Cl. A61K 31/4745; A61K 31/5377; A61K 31/557; A61K 31/4178; A61K 31/137

(52) U.S. Cl. 514/292; 514/232.5; 514/573; 514/397; 514/649

(57) ABSTRACT

Methods and compositions used for lowering intraocular pressure. More particularly, the methods and compositions for lowering intraocular pressure pertain to the use of at least a phenanthroline derivative in an ophthalmic delivery solution.

### RELATED APPLICATIONS

This application claims priority to U.S. Provisional Patent Application, Ser. No. 60/629,786, entitled “Phenanthroline and Derivatives Thereof Used to Lower Intraocular Pressure in an Affected Eye,” filed on Nov. 19, 2004, having Dibas, et al., listed as the inventor(s), the entire content of which is hereby incorporated by reference.

### STATEMENT OF RIGHTS TO INVENTIONS MADE UNDER FEDERALLY SPONSORED RESEARCH

The government may own certain rights in the present invention pursuant to grant number EY11979 from NIH/NEI and 009768-018 from Advanced Research Program-Texas.

### BACKGROUND

The present invention pertains generally to methods and compositions for lowering intraocular pressure in an affected eye. More particularly, the invention pertains to the use of a phenanthroline derivative in an topical ophthalmic delivery solution for the lowering intraocular pressure in an affected eye.

Glaucoma is a group of diseases that can damage the eye's optic nerve and result in vision loss and blindness. However, with early diagnosis and treatment, a patients eye's can often be protected from serious vision loss. The front of the eye comprises a space called the anterior chamber. The clear fluid, called aqueous humor (“AH”), which flows in and out of the chamber continuously nourishes nearby tissues. The fluid leaves the anterior chamber at the open angle where the cornea and iris meet. When the fluid reaches the angle, it flows through a spongy meshwork, like a drain, and leaves the eye.

However, if fluid that reaches the angle passes too slowly through the meshwork drain, fluid will build up and the pressure inside the eye rises to a level that may damage the optic nerve. Increased eye pressure is a risk factor for glaucoma, but is not a defining factor of the disease. A person is considered to have glaucoma only if the optic nerve is damaged. If a patient has increased eye pressure but does not have damage to the optic nerve, the patient is not considered to have glaucoma. However, when the optic nerve of the patient is damaged from increased pressure, open-angle glaucoma and vision loss may result, which is the reason controlling pressure inside the eye is important.

Glaucoma cannot be cured, but it can be treated by one of two ways: medication or surgery. Both of these treatments are aimed at lowering intraocular pressure. In the U.S., medications are considered to be the first-line treatment for the disease. If this fails, then surgery will be considered.

Medication

Glaucoma medications are either oral or topical. Topical medications such as eye drops, eye ointments, or inserts (strips of medication inserted in the corner of the eye), work to reduce IOP either by increasing the outflow of fluid from the eye or by reducing the amount of fluid produced by the eye. There are five general types of topical medications that achieve these purposes:
I. Miotics are used to increase the outflow of fluid. Some products include: IsoptoCarpine®, Ocusert®, Pilocar®, and Pilopine®.
II. Epinephrines are used to increase the outflow of fluid. Some products include Epifrin® and Propine®
III. Beta-Blockers are used to reduce the amount of fluid. Some products include Betagan®, Betimol®, Betoptic®, Ocupress®, Optipranalol®, and Timoptic®.
IV. Carbonic Anhydrase Inhibitors and Alpha-Adrenergic Agonists are used to reduce the amount of fluid. Some products include: Alphagan® (brimonidine), Iopidine® (apraclonidine), and Trusopt®.
V. Prostaglandin Analogs are used to increase the outflow of fluid through a secondary drainage route. Some products include: Lumigan®, Rescula®, Travatan®, and Xalatan® (latanoprost).

The most common type of oral medication are Carbonic Anhydrase Inhibitors, which include Daranide®, Diamox®, and Neptazane®.

Glaucoma patients are typically started with one or a combination of medications. If a patient does not respond to one type of drug, he or she can be switched to another medication or combination of medications until all possibilities have been exhausted. Once this happens, the ophthalmologist may recommend surgery.

Glaucoma Surgery

Patients that have been treated with medication, but who still have an elevated IOP, may be recommended for either laser or conventional surgery by an ophthalmologist. There are generally three types of laser glaucoma surgery that can be performed:
I. Trabeculoplasty—the use of a laser to burn tissue from the trabecular meshwork (a structure within the eye that controls the flow of fluid), which increases the outflow of fluid from the eye. This type of laser surgery is used to treat patients with open-angle glaucoma.
II. Iridotomy—the use of a laser to burn tissue from the iris, which increases the outflow of fluid from the eye. This type of laser surgery is used to treat patients with closed-angle glaucoma.
III. Cyclophotocoagulation—the use of a laser to burn ciliary tissue, which decreases the production of fluid in the eye. This type of laser surgery is used to treat patients who have failed to respond to other types of surgery.

If laser surgery fails to lower IOP, the surgeon may recommend conventional surgery, known as trabeculectomy or filtering surgery. This is an outpatient procedure involving the removal of a tiny piece of the eye under the eyelid. This creates a new drainage path that increases the outflow of fluid from the eye.

Optic Nerve Head Damage

The present-day drugs and surgery to treat glaucoma are limited by their actions as they mitigate only the major symptom of the disease, which is elevated intraocular pressure due to blockage of the outflow pathway as seen in primary open angle glaucoma. These drugs do not target the site of damage i.e. prevent the onset of damage to the optic nerve head and consequently do not prevent retinal ganglion cell death and optic nerve damage in glaucoma. Once initiated, the glaucomatous damage to the retinal ganglion cells occurs in a gradual yet progressive manner despite lowering the pressure. Moreover, these drugs only treat one form of glaucoma, primary open angle glaucoma (“POAG”) in which elevated intraocular pressure (“IOP”) may be a major symptom/cause for retinal ganglion cell death. However, in other glaucoma, like normal tension glaucoma (“NTG”), IOP is within the normal range of 15-20 mm Hg, yet the damage to the optic nerve and progression of retinal ganglion cell death is identical to that seen in POAG patients.

Although not wanting to be bound by theory, compositions that can directly target the source of damage and avert the potential cause of retinal ganglion cell death and optic nerve damage from occurring offer advantages over conventional medications. Additionally, such compounds can prevent glaucomatous damage to the optic nerve irrespective of the etiology of the disease, as seen in different forms of glaucoma (e.g. POAG and NTG).

Miotics

Miotics are drugs that cause constriction of the pupil and increase drainage of aqueous. Pilocarpine (Isopto Carpine), the principal miotic used in glaucoma therapy, was isolated from the leaves of Pilocarpus plants in the 19th century. It was first used for glaucoma therapy in 1956. Miotics (acetylcholine agonists and cholinesterase inhibitors) are thought to promote increased trabecular aqueous outflow by contracting the ciliary muscle of the eye.

Side effects such as accommodative spasm, brow-ache and myopia are more pronounced in younger patients who are treated with miotics. In patients with cataracts, miotics may contribute to functional disability by decreasing daytime and, perhaps more significantly, nighttime vision. Systemic cholinergic effects such as nausea, vomiting, sweating and cutaneous vasodilatation may occur.

Pilocarpine therapy is relatively inexpensive. Nevertheless, the high incidence of ocular side effects and the inconvenience of dosing four times daily make pilocarpine less popular than other agents used in the medical management of glaucoma. Pilocarpine in a continuous-release vehicle (Ocusert Pilo) applied once weekly to the lower conjunctival sac is promising in theory but has not gained popularity, in part because it tends to fall out of the eye.

Sympathomimetics

Topical sympathomimetics may be divided into epinephrine (alpha- and beta-receptor stimulation) and clonidine-like agents (alpha-receptor stimulation). Sympathomimetics either decrease aqueous production or increase aqueous outflow. Epinephrine has frequent ocular allergic side effects and consequently is less commonly used in patients with glaucoma. Dipivefrin (Propine), an epinephrine prodrug, is taken twice daily. Although dipivefrin produces fewer ocular and systemic side effects than epinephrine, it is being supplanted by clonidine-like agents for glaucoma therapy.

The FDA has labeled apraclonidine (lopidine) for use in the management of transient IOP elevations after ocular surgery. This agent is associated with a high incidence of tachyphylaxis (loss of effect) and clonidine-like central nervous system (CNS) effects such as somnolence and orthostasis. Ocular allergic side effects are common. Thus, apraclonidine has only limited use in the chronic management of primary open-angle glaucoma.

Brimonidine (Alphagan) is approved for maintenance glaucoma treatment and may be suitable as monotherapy. This drug has fewer CNS and ocular side effects than apraclonidine. The increased selectivity of brimonidine for alpha2-receptor sites is postulated to decrease IOP by limiting aqueous production and facilitating increased outflow via the uveoscleral pathway. Tachyphylaxis occurs less frequently with brimonidine than with apraclonidine. Potential limitations to the use of brimonidine include its dosing schedule (two or three times daily) and its cost. Coadministration with monoamine oxidase inhibitors is contraindicated because of the risk of precipitating a hypertensive crisis.

U.S. Pat. No. 3,809,714 issued to Hussain et al., on May 7, 1974 titled “Novel Ester of [Methylamino)Methyl] Benzyl Alcohol,” (“the '714 Patent”), is an example of a pro-drug of epinephrine that is used in the treatment of glaucoma. The entire content of which is herein incorporated by reference. As mentioned above, epinephrines are used to increase the outflow of fluid.

Beta Blockers

Topically administered beta blockers have been the mainstay of glaucoma therapy for more than two decades. Timolol maleate (Timoptic) is the standard agent against which other medications are measured in terms of efficacy, side effects and cost. Beta blockers are thought to lower IOP mainly by decreasing aqueous humor production in the ciliary body of the eye. They may also induce a slight increase in aqueous outflow.

Although topically administered timolol is frequently recommended as first-line therapy, the actions and side effects of this drug may limit its use. Timolol and other topically applied beta blockers have been associated with asthma exacerbation, including status asthmaticus, worsening congestive heart failure, heart block and, rarely, sudden death. Because these agents may block the typical systemic manifestations of hypoglycemia, they should be used with caution in patients with diabetes mellitus.

Central beta blockade from ocular application of these agents may also result in dysthymia or frank depression, as commonly occurs with orally administered beta blockers. Impotence is another well recognized side effect of topically applied beta blockers. This adverse effect may cause patients to stop using these medications. Such patients may be reluctant to discuss this discontinuation or the reason behind it with their physician.

Betaxolol (Betoptic), a cardioselective beta blocker, has a more favorable cardiopulmonary side effect profile than timolol. Because timolol has a superior IOP-lowering effect, it is frequently recommended over betaxolol when cardiopulmonary compromise is not of concern. Nevertheless, several studies 18-20 demonstrate that betaxolol provides superior visual field preservation. Betaxolol is marketed in a suspension (Betoptic S) with a lower medication concentration and a reportedly decreased incidence of systemic side effects compared with the corresponding solution.

Other topically applied beta blockers include metipranolol (Optipranolol), carteolol (Ocupress) and levobunolol (Betagan). The manufacturer of carteolol claims that the drug has an intrinsic sympathomimetic effect; therefore, it theoretically may have fewer cardiopulmonary side effects than timolol. Beta blockers are typically applied twice daily, although once-daily therapy may be effective in some patients. A recently introduced gel-forming solution of timolol maleate (Timoptic-XE) has the advantage of once-daily dosing. This solution is likely to become the therapy of choice in patients who can tolerate beta blockers.

Carbonic Anhydrase Inhibitors and Alpha-Adrenergic Agonists

Orally administered carbonic anhydrase inhibitors have long been used in the management of primary open-angle glaucoma refractory to other forms of medical therapy. Agents such as acetazolamide (Diamox) and methazolamide (Neptazane) decrease aqueous humor secretion by the ciliary epithelium.

The use of carbonic anhydrase inhibitors is limited by side effects ranging from general malaise to symptomatic metabolic acidosis, renal calculi and bone marrow suppression. Orally administered carbonic anhydrase inhibitors may accentuate the effects of diuretics and contribute to volume depletion and clinically significant hypokalemia. Concomitant use with aspirin increases the risk of salicylate toxicity.

Dorzolamide (Trusopt) and brinzolamide (Azopt) are the first topical carbonic anhydrase inhibitors labeled by the U.S. Food and Drug Administration (FDA) for the treatment of primary open-angle glaucoma. Each drug is used two to three times daily. Dorzolamide is also marketed in combination with timolol (Cosopt). These agents are favored over oral agents because of their greater site specificity and markedly fewer systemic side effects.

Acetazolamide, dorzolamide and brinzolamide are sulfonamide derivatives. As such, the potential exists for bone marrow dyscrasias, transaminitis and dermatologic reactions ranging from simple hypersensitivity to Stevens-Johnson syndrome. To date, however, the topical agents have not been associated with these adverse effects. Dorzolamide and brinzolamide should not be used in patients with a history of hypersensitivity to sulfa medications, and their use is not recommended in patients with moderate to severe renal impairment.

Established systemic side effects associated with topically administrated dorzolamide and brinzolamide include bitter taste (experienced by up to 25 percent of patients), headache, nausea, asthenia and fatigue. Rarely, nephrolithiasis may occur. The manufacturer claims a lower incidence of ocular side effects for brinzolamide. However, added experience with brinzolamide is required to determine if this stated advantage is supported by repeated clinical use.

U.S. Pat. No. 4,517,199 issued to York, et al., on May 14, 1985, titled “Method for Lowering Intraocular Pressure using Phenylimino-Imidazoles,” (“the '199 Patent”), discloses carbonic anhydrase inhibitor compounds for lowering intraocular pressure. The entire content of which is herein incorporated by reference.

Prostaglandin Analogs

Latanoprost (Xalatan) was recently labeled for use in patients with glaucoma. This agent is one of the prostaglandin analogs, a new class of agents for the treatment of glaucoma. Latanoprost is taken once daily at bedtime. IOP reduction is equivalent to that achieved with twice-daily timolol therapy. Compared with timolol, latanoprost has a more favorable local and systemic side effect profile.

The development of a prostaglandin suitable for clinical use in the treatment of glaucoma was previously hampered by the prevalence of ocular side effects, primarily conjunctival hyperemia. Like dipivefrin, latanoprost (a prostaglandin F2alpha analog) is a prodrug that produces the desired clinical effect with a more tolerable degree of ocular side effects.

Latanoprost lowers IOP by increasing uveoscleral outflow (the minor pathway for the removal of aqueous humor from the anterior chamber of the eye). Interestingly, latanoprost reduces IOP to a greater extent when it is administered once daily in the evening than when it is applied either once daily in the morning or twice daily. Unlike timolol, latanoprost exhibits a sustained IOP-lowering effect throughout the day and night. Increased iris pigmentation occurs in up to one in six patients treated with latanoprost and is the main focus of discussions regarding the side effect profile of this drug. Patients with mixed-color irises (i.e., brown-gray or brown-green) are most at risk for this side effect, which is the result of increased melanin production. Because melanocyte division is not stimulated, the color change is not believed to place the patient at added risk for melanoma. The color change is stable and may not be reversible with discontinuation of the drug. Long-term effects are unknown. Lash growth, another documented ocular side effect of latanoprost, is thought to be of only cosmetic significance. In case reports, latanoprost has also been associated with iritis, hypotony with choroidal effusion, and cystoid macular edema.

Phenanthroline

One embodiment of the current invention is a method of lowering intraocular pressure using a composition of phenanthroline or derivative thereof. One general structure for phenanthroline and derivatives is shown in structural formula I: 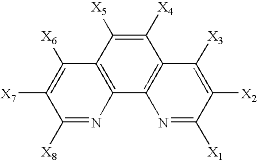
wherein X₁-X₈ are the same or different and comprise H, OH, O, CₙH₂ₙ₊₁ (wherein n=1-4), phenyl or substituted phenyl, Cl, —NO₂, or —CN. As discussed herein, phenanthroline and derivatives thereof can lower IOP. Three clinical trials have confirmed that lowering the IOP in patients with glaucoma will decrease or prevent the onset of the disease. More specifically, the Ocular Hypertension Treatment Study, the Early Manifest Glaucoma Trial and the Collaborative Initial Glaucoma Treatment Study demonstrated that lowering IOP by at least by 20% is likely to avoid progression of the disease and thus is neuroprotective. Thus, targeting reduction of IOP remains a viable and important part of the therapeutic drug development. Consequently, phenanthroline and derivatives thereof are effective for open angle glaucoma, and normal tension glaucoma.

Phenanthroline can be prepared in a solution to be utilized as a topically applied medication. Topically applied phenanthroline will minimize side effects that may be seen by systemic injection of other IOP lowering drugs.

### SUMMARY

The present invention pertains generally to methods and compositions for lowering intraocular pressure in an affected eye. More particularly, the invention pertains to the use of a phenanthroline derivative in a topical ophthalmic delivery solution for the lowering intraocular pressure in an affected eye.

A first aspect of the present invention is a topical ophthalmic composition useful for the lowering intraocular pressure in an affected eye. The topical ophthalmic composition comprises a pharmaceutically effective amount of at least one phenanthroline derivative in a carrier suitable for topical ophthalmic delivery. One specific embodiment comprises the phenanthroline derivative having a general structure of Formula I: 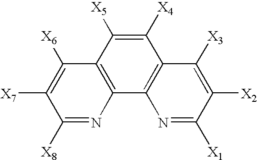
wherein X₁-X₈ are the same or different and comprise H, OH, O, CₙH₂ₙ₊₁ (wherein n=1-4), phenyl or substituted phenyl, Cl, —NO₂, or —CN. Examples include: 1,10-phenanthroline; 1,10-phenanthroline-5-acetonitrile; 2,9,dimethyl-1-10-phenanthroline; 3,4,7,8-tetramethyl-1-10-phenanthroline; 4,7,-dihydroxy-1-10-phenanthroline; 4,7,-dimethyl-1,10-phenanthroline; 4,7-diphenyl-1,10-phenanthroline; 4-methyl-1,10-phenanthroline; 5,6-dimethyl-1,10-phenanthroline; 5,6-dimethyl-1,10-phenanthroline; 5-chloro-1,10-phenanthroline; 5-methyl-1,10-phenanthroline; 5-nitro-1,10-phenanthroline; and their pharmaceutically acceptable analogues and derivatives. The final composition concentration of the phenanthroline derivative is in a range of about 0.05 and about 1.5 wt %. Suitable carriers for topical ophthalmic delivery include: anionic, mucomimetic polymers; gelling polysaccharides; finely-divided drug carrier substrates; and combinations thereof.

A second aspect of the current invention is a method of lowering intraocular pressure in an affected eye, comprising applying to the affected eye a pharmaceutically effective amount of at least one phenanthroline derivative in a carrier suitable for topical ophthalmic delivery. A preferred topical ophthalmic composition comprises a pharmaceutically effective amount of 1,10-phenanthroline in a carrier suitable for topical ophthalmic delivery, as described above.

The methods of lowering intraocular pressure in an affected eye further comprise contacting compounds described above in combination with at least another composition used for treating glaucoma. The combination of at least one phenanthroline derivative and a second composition selected from a group that include, but is not limited to, β-blockers, prostaglandins, α₂ agonists, and miotics. Commercially available examples of the second compound comprise: Isopto®Carpine (Pilocarpine ophthalmic), Epifrin® (epinephrine ophthalmic), Propine® (dipivefrin ophthalmic), Betagan® (levobunolol ophthalmic), Betimol® (timolol ophthalmic), Betoptic® (betaxolol ophthalmic), Ocupress® (carteolol ophthalmic), Timoptic® (timolol ophthalmic), Alphagan® (brimonidine), Iopidine® (apraclonidine ophthalmic), Trusopt® (dorzolamide ophthalmic), Lumigan® (bimatoprost ophthalmic), Rescula® (unoprostone ophthalmic), Travatan® (travoprost ophthalmic), Xalatan® (latanoprost ophthalmic), Daranide(® (dichlorphenamide), Diamox® (acetazolamide), or Neptazane® (methazolamide).

Ophthalmic products are typically packaged in multidose form (2-15 ml volumes). Preservatives may be required to prevent microbial contamination during use. Suitable preservatives include: benzalkonium chloride, chlorohexidine, thimerosal, chlorobutanol, methyl paraben, propyl paraben, phenylethyl alcohol, edetate disodium, sorbic acid, polyquatemium-1, or other agents known to those skilled in the art. Some of these preservatives, however, may be unsuitable for particular applications (e.g., benzalkonium chloride may be unsuitable for intraocular injection or interference of preservatives with phenanthroline(s). Such preservatives are typically employed at a level of from is about 0.001% to 1.0% weight/volume (“w/v”).

Topical administration of phenanthrolines comprises a dosage generally in a range between about 0.001% and 5% weight/volume (“w/v”), preferably between 0.25% and 2.5% (w/v), and more preferably at about 1% (w/v). Solutions, suspensions, ointments, gels, jellies and other dosage forms adapted for topical administration are preferred. Similar dose ranges and effective doses as that for topical administration will be employed for the gel preparations. Additionally, phenanthrolines may be delivered slowly, over time, to the afflicted tissue of the eye through the use of contact lenses. This regimen is generally performed by first soaking the lenses in a solution containing phenanthrolines and then applying the contact lenses to the eye for normal wear.

As used herein, the term “pharmaceutically acceptable carrier” refers to any formulation which is acceptable, i.e., safe and provides the appropriate delivery for the desired route of administration, of an effective amount of at least one phenanthrolines of the present invention.

The compositions of the present invention are further illustrated in the following formulation examples, phenanthrolines of the present invention are represented generically in the examples as “phenanthroline”. However, the drugs listed in Tables 1 and Table 2 are representative agents in these classes. The invention includes any agent related in structure and pharmacology to these agents. These agents will be prepared for use in therapeutic effective concentrations for the treatment of ocular disease that result in elevated intraocular pressure of an affected eye (e.g. glaucoma). According to the present invention, a therapeutically effective amount of phenanthroline is an amount sufficient to relieve or prevent elevated intraocular pressure of an affected eye. Dosages can be readily determined by one of ordinary skill in the art and can be readily formulated into pharmaceutical dosing entities (i.e. pills, gels, drops, etc.).

Phenanthroline(s) may be administered topically, by intracameral injection, periocular injection or transcleral administration. The exact dosage of one or more phenanthrolines to be administered to the patient will vary, but will be determined by clinicians skilled in the art. Various factors affecting the dosage amount include the actual disease to be treated, the severity of condition, the health of the patient, the potency and specific efficacy of the phenanthrolines, and so on. The amount dosed, however, will be an “effective amount.”

The phenanthrolines of the present invention may be contained in various types of ophthalmic compositions, in accordance with formulation techniques known to those skilled in the art For example, the compounds may be included in solutions, suspensions and other dosage forms adapted for topical use.

The ophthalmic composition of the present invention will include one or more phenanthrolines of the present invention and a pharmaceutically acceptable vehicle or carrier. Aqueous solutions are generally preferred, based on ease of formulation and physiological compatibility. However, the phenanthrolines of the present invention may also be readily incorporated into other types of compositions, such as suspensions, viscous or semi-viscous gels or other types of solid or semi-solid compositions. The ophthalmic compositions of the present invention may also include various other ingredients, such as buffers, preservatives, co-solvents and viscosity building agents. Examples of carrier solutions suitable for topical ophthalmic delivery comprises: anionic, mucomimetic polymers; gelling polysaccharides; finely-divided drug carrier substrates; mineral oil; liquid petrolatum; white petrolatum; propylene glycol; polyoxyethylene; polyoxypropylene compound; emulsifying wax and water; or combinations thereof.

### BRIEF DESCRIPTION OF THE DRAWINGS

FIG. 1 shows the three general structures (I-III) for phenanthroline;

FIG. 2 shows specific examples of phenanthroline derivatives suitable for use as treatment agents;

FIG. 3 shows specific examples for phenanthroline derivatives suitable for use as treatment agents;

FIG. 4 shows 1% Phenanthroline used to lower intraocular pressure in Cohotr 2-4-04;

FIG. 5 shows 1% Phenanthroline used to lower intraocular pressure with ID#1-17-48 in Cohort 2-4-04; and

FIG. 6 shows the effect of specific Phenanthroline derivatives on lowining IOP in the Morrison Model of glaucoma: Panel (A) about 1% 1,7-Phenanthroline; Panel (B) about 4,7-dihydroxy-1,10-phenanthroline; and Panel (C) about 1,10-phenanthroline5,6-dione.

### DETAILED DESCRIPTION OF THE PREFERRED EMBODIMENTS

The activation of metallo-proteinases (“MMPs”) has been suggested to lower IOP in glaucoma. The entirety of each of the following citations are herby incorporated by reference: Mamiya K., et al., in Exp Eye Res. 2004 September; 79(3):405-10, (“the Mamiya '405 Reference”); Dan J. A., in Arch Ophthalmol. 2002 May; 120(5):548-53, (“the Dan '53 Reference”); and U.S. Pat. No. 5,260,059 issued to Acott on Nov. 9, 1993, titled “Treatment of Open-Angle Glaucoma by Modulation Matrix Metalloproteinases and their Inhibitor,” (“the '059 Patent”).

The Mamiya '405 Reference indicated that trabeculectomy following MMP-3 transfection in rabbit eye caused significantly decreased levels of IOP in comparison with controls (trabeculectomy alone or trabeculectomy following vector transfection). The Dan '53 Reference indicated that injection of purified MMPs into eyes of glaucoma patients caused a reduction in IOP. Similarly, claims of the '059 Patent are drawn to methods for treating open-angle glaucoma in an eye of a subject by delivering an effective amount of MMPs (e.g. MMP-1, MMP-2 or MMP-3). Thus, each of these references indicated that the activation of metallo-proteinases (“MMPs”) in an effected eye will lower IOP.

The phenanthroline compositions and methods of this invention lowers IOP in an affected eye, however, the mechanism of action of phenanthroline in lowering IOP is not completely understood. Although not wanting to be bound by theory, phenanthroline is believed to be reversible inhibitor of MMPs, such as thermolysin (Ki=40 μM for thermolysin), which teaches away from the activation model of MMPs that was proposed previously.

Elevated IOP can produce nitric oxide through induction and subsequent expression of Nitric Oxide Synthase-2 (“NOS-2”), which in turn may damage the optic nerve. Although not wanting to be bound by theory, the lowering IOP by phenanthroline may prevent the expression of NOS-2 and provide neuroprotection through this mechanism. Three clinical trials have confirmed that lowering the IOP in patients with glaucoma will decrease or prevent the onset of the disease. More specifically, the Ocular Hypertension Treatment Study, the Early Manifest Glaucoma Trial and the Collaborative Initial Glaucoma Treatment Study demonstrated that lowering IOP by at least by 20% is likely to avoid progression of the disease and thus is neuroprotective. Thus, targeting reduction of IOP remains a viable and important part of the therapeutic drug development.

The following examples are provided to further illustrate this invention and the manner in which it may be carried out. It will be understood, however, that the specific details given in the examples have been chosen for purposes of illustration only and not be construed as limiting the invention.

### EXAMPLE 1

### Phenanthroline Compositions to Lower IOP

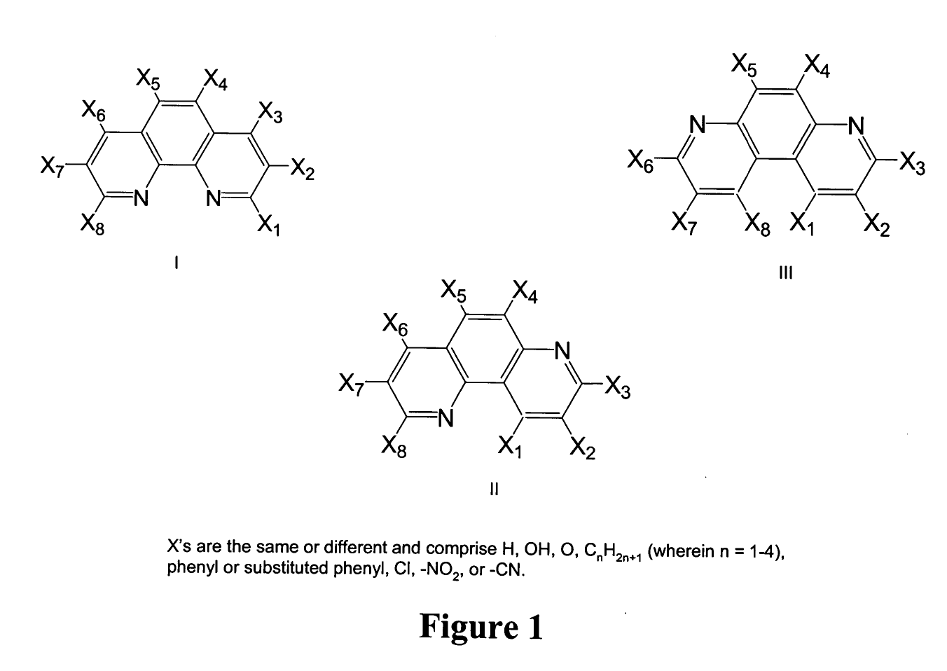

Three isomers of phenanthroline are shown in the Formula I, II and III in FIG. 1. Formula I comprises: 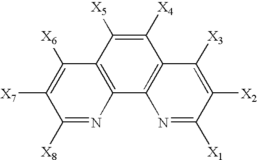
wherein X₁-X₈ are the same or different and comprise H, OH, O, CₙH₂ₙ₊₁ (wherein n=1-4), phenyl or substituted phenyl, Cl, —NO₂, or —CN.

Formula II comprises: 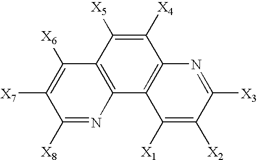
wherein X₁-X₈ are the same or different and comprise H, OH, O, CₙH₂ₙ₊₁ (wherein n=1-4), phenyl or substituted phenyl, Cl, —NO₂, or —CN.

Formula III comprises: 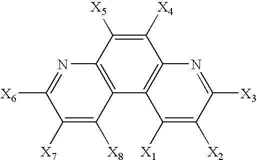
wherein X₁-X₈ are the same or different and comprise H, OH, O, CₙH₂ₙ₊₁ (wherein n=1-4), phenyl or substituted phenyl, Cl, —NO₂, or —CN.

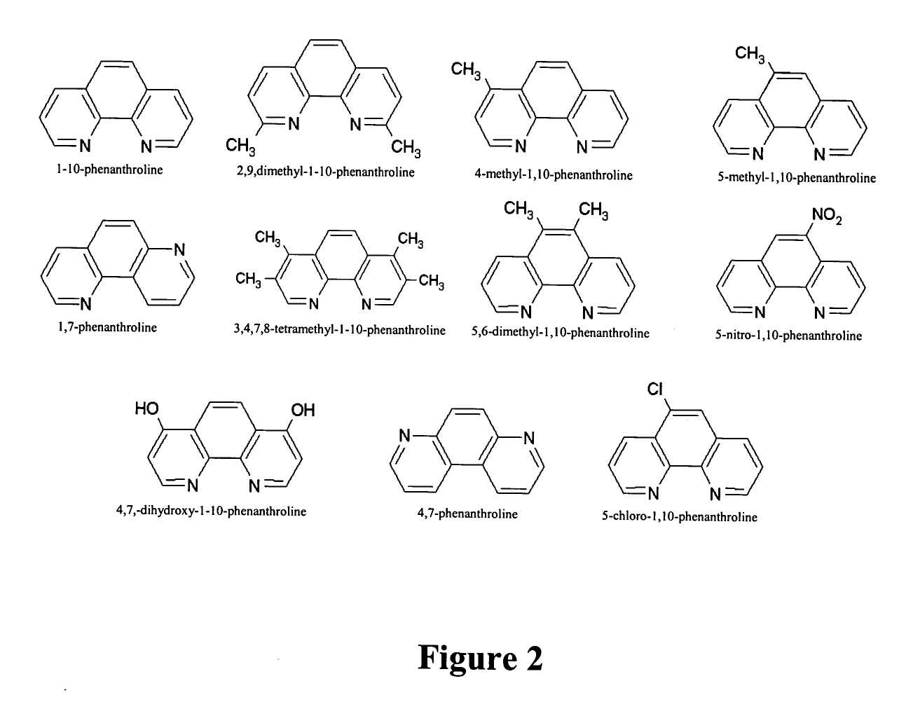

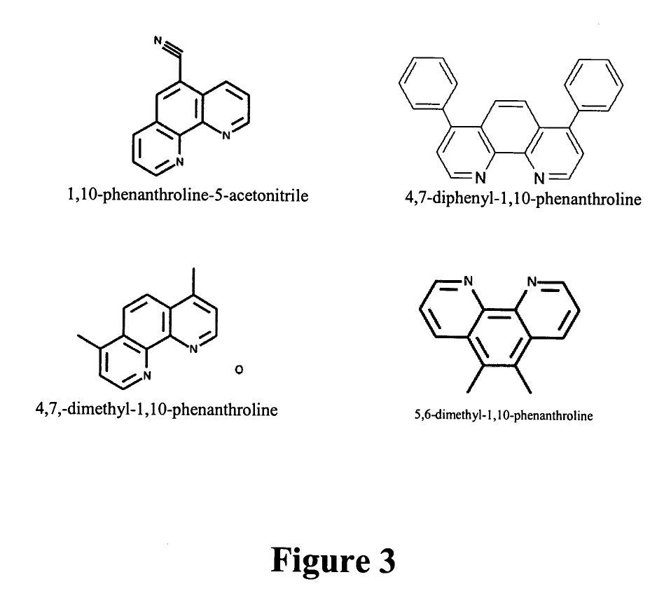

It should be apparent to one of ordinary skill in the art that many derivatives of phenanthroline can be used in formulations for treatment of lowering IOP that do not depart from the spirit or scope of the present invention. Certain embodiments of this invention include the use of the specific chemical structures and derivatives of phenanthroline that are shown in FIGS. 2-3.

### EXAMPLE 2

### The Morrison Rat Model for Glaucoma

The Intraocular Pressure (“IOP”) in a rat eye was lowered following treatment of the eye with about a 1% (w/v) phenanthroline solution. Briefly, the IOP was elevated in one eye for rats grouped as specified below by using the method as described by Morrison et al (1997). The elevation of IOP was achieved in one eye as described by Morrison et al. (1997), wherein 50 μl of 1.8M saline was injected into the episcleral veins of one eye of anesthetized rats such that blanching was observed. The other eyes was used as a control. Rats were housed post-surgically in constant low light (<90 lux) to minimize effects of circadian influences on IOP. Five rats were used per treatment group.

A composition of 1,10-phenanthroline (20-25 μl of a 1% (w/v) solution in saline (0.9 % NaCl) was applied topically on the Morrison rat model eye, wherein the IOP of the treated eye was measured after 1 hour and 6 hours using a tonometer as described in Morrison J C, et al., Exp Eye Res 64, 85-96. 1997. The 1,10-phenanthroline use for these data points was purchased from Sigma Chemical (St. Louis, Mo.).

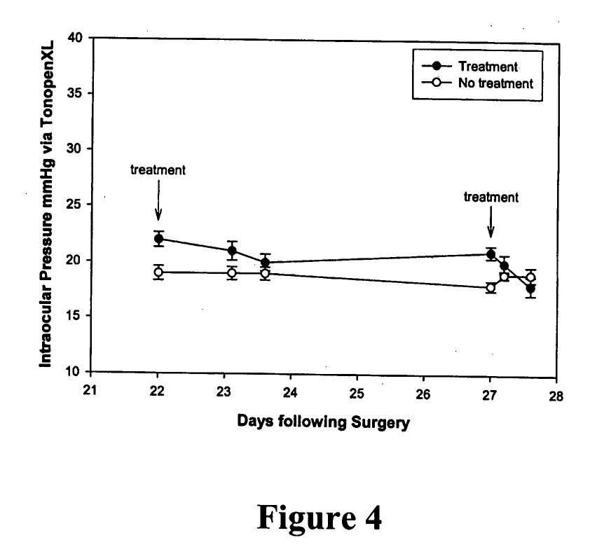

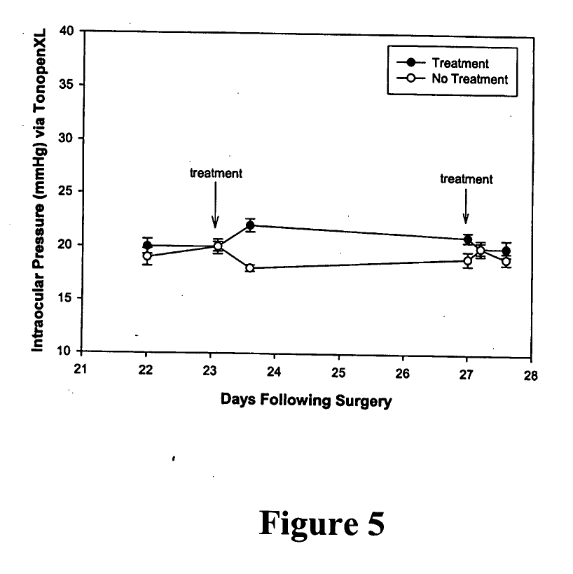

As shown in FIG. 4, the intraocular pressure was decreased in the treatment group following treatment of 1% phenanthroline at 22 and 27 days following surgery in cohort 2-4-04. FIG. 5 shows that the intraocular pressure was decreased in the treatment group following treatment of 1% phenanthroline at 27 days following surgery in cohort 2-4-04.

Although the 1% phenanthroline solution was applied once topically, the treatment may be used in a treatment regimen similar to current glaucoma therapeutics. The exact dosage of one or more phenanthroline compositions to be administered to a patient will vary, but will be determined by clinicians skilled in the art. Various factors affecting the dosage amount include the actual disease to be treated, the severity of condition, the health of the patient, the availability of the active drug at the retina, potency and specific efficacy of the specific phenanthroline composition, and so on. The amount dosed, however, will be an “effective amount”. As used herein, the term “effective amount” is an amount, which lowers IOP to a level that is effective for therapy. For example, a preferred embodiment would includes treating each affected eye having an elevated IOP with about 1-5 drops of about 1% phenanthroline at about 1-4 times daily. Additionally, phenanthroline compositions may be administered topically, by intracameral injection, periocular injection or transcleral administration.

The phenanthroline compositions of the present invention may be contained in various types of ophthalmic compositions, in accordance with formulation techniques known to those skilled in the art. For example, the compounds may be included in solutions, suspensions and other dosage forms adapted for topical or intracameral use.

The ophthalmic compositions of the present invention will include one or more phenanthroline compositions of the present invention and a pharmaceutically acceptable vehicle. Aqueous solutions are generally preferred, based on ease of formulation and physiological compatibility. However, the phenanthroline compositions of the present invention may also be readily incorporated into other types of compositions, such as suspensions, viscous or semi-viscous gels or other types of solid or semi-solid compositions. The ophthalmic compositions of the present invention may also include various other ingredients, such as buffers, preservatives, co-solvents and viscosity building agents. The preferred active doses of phenanthroline compositions that will be employed for topical application will range from about 0.1% to about 2.5% (w/v).

An appropriate buffer system (e.g., hydrochloric acid/sodium hydroxide, sodium phosphate, sodium acetate or sodium borate) may be added to prevent pH drift under storage conditions.

Ophthalmic products are typically packaged in multidose form (2-15 ml volumes). Preservatives may be required to prevent microbial contamination during use. Suitable preservatives include: benzalkonium chloride, chlorohexidine, thimerosal, chlorobutanol, methyl paraben, propyl paraben, phenylethyl alcohol, edetate disodium, sorbic acid, polyquatemium-1, or other agents known to those skilled in the art. Some of these preservatives, however, may be unsuitable for particular applications (e.g., benzalkonium chloride may be unsuitable for intraocular injection or interference of preservatives with phenanthroline compositions). Such preservatives are typically employed at a level of from is 0.001 to 2.5% weight/volume (“w/v”).

Topically administered drugs used to lower IOP in glaucoma may be able to gain access to the retina. However, there are no effective methods to directly target the back of the eye for chronic conditions, but it is presently contemplated that such methods will be eventually developed, wherein delivery of phenanthroline to the back of the eye to lower IOP are considered. Topical administration of phenanthroline compositions will generally range between about 0.001% to about 2.5% weight/volume (“w/v”), preferably between about 0.5% and about 1.5% (w/v). Solutions, suspensions, ointments, gels, jellies and other dosage forms adapted for topical administration are preferred. Similar dose ranges and effective doses as that for topical administration will be employed for the gel preparations. Additionally, phenanthroline compositions may be delivered slowly, over time, to the afflicted tissue of the eye through the use of contact lenses. This regimen is generally performed by first soaking the lenses in a solution containing a phenanthroline composition and then applying the contact lenses to the eye for normal wear.

As used herein, the term “pharmaceutically acceptable carrier” refers to any formulation which is acceptable, i.e., safe and provides the appropriate delivery for the desired route of administration, of an effective amount of at least one phenanthroline compositions of the present invention. For example, a carrier suitable for topical ophthalmic delivery may comprise: anionic, mucomimetic polymers; gelling polysaccharides; finely-divided drug carrier substrates; and combinations thereof. Carriers for topical administration of the compounds of this invention include, but are not limited to, mineral oil, liquid petrolatum, white petrolatum, propylene glycol, polyoxyethylene, polyoxypropylene compound, emulsifying wax and water.

The invention includes compositions related in structure and pharmacology to these agents, for example see FIGS. 1-3. These agents will be prepared for use in therapeutic effective concentrations for the treatment of ocular disease (e.g. glaucoma). According to the present invention, a therapeutically effective amount of a phenanthroline composition is an amount sufficient to relieve or prevent optic nerve damage. Dosages can be readily determined by one of ordinary skill in the art and can be readily formulated into pharmaceutical dosing entities (i.e. drops, pills, gels, etc.).

### EXAMPLE 3

Topical ophthalmic composition useful for treating ocular neural tissue:

| Component | % w/v |
|---|---|
| Phenanthroline | 0.001-2.5 |
| Dibasic Sodium Phosphate | 0.2 |
| HPMC | 0.5 |
| Polysorbate 80 | 0.05 |
| Benzalkonium Chloride | 0.01 |
| Sodium Chloride | 0.75 |
| Edetate Disodium | 0.01 |
| NaOH/HCl | q.s., pH 7.4 |
| Purified Water | q.s. 100% |

### EXAMPLE 4

A sterile solution useful for treating ocular neural tissue:

| Component | % w/v |
|---|---|
| Phenanthroline | 0.001-2.5 |
| Cremophor EL .RTM. | 10 |
| Tromethamine | 0.12 |
| Mannitol | 4.6 |
| Disodium EDTA. | 0.1 |
| Hydrochloric acid or | q.s., pH to 7.4 |
| Water for injection | q.s. 100% |

### EXAMPLE 5

A tablet formulation useful for treating ocular neural tissue:

| | Amount per tablet | |
|---|---|---|
| Ingredient | (mg) | |
| Phenanthroline | 70-280 | mg/kg/day |
| Cornstarch | 50 | |
| Lactose | 145 | |
| Magnesium stearate | 5 | |

### EXAMPLE 6

A solution useful for treating ocular neural tissue:

| Ingredient | Amount |
|---|---|
| Phenanthroline | 70-280 mg/kg/day |
| 0.4 M KH₂PO₄ | 2.0 ml |
| 1 N KOH solution q.s. | to pH 7 . . . 0 |
| Water for injection | q.s. to 20 ml |

### EXAMPLE 7

### Using 1,7-Phenanthroline to Lower IOP in the Morrison Model of Glaucoma

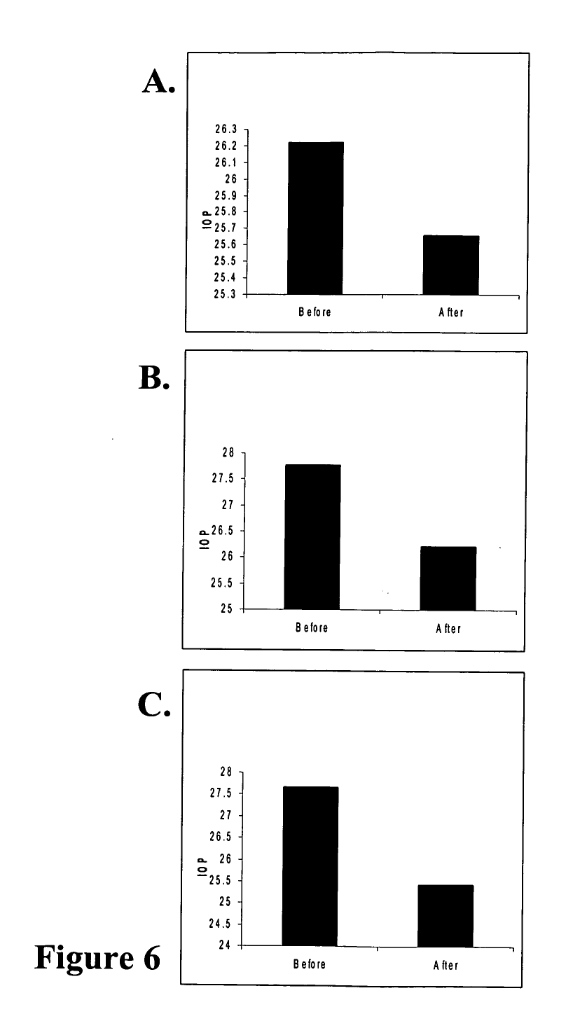

A solution of 1,7-phenanthroline was prepared by dissolving 10 mg of 1,7-phenanthroline in 32 μl methanol (100%) forming a concentrated 1,7-phenanthroline solution. About 3 μl of the concentrated 1,7-phenanthroline solution was then added to 96 μl of saline (0.9% NaCl) to form a working 1,7-phenanthroline solution. The working 1,7-phenanthroline solution was then used in the eye of the Morrison Model of Glaucoma. The average intraocular pressure (IOP) in Morrison model eye was 26.22 (average of nine readings) prior to application of the working 1,7-phenanthroline solution. In contrast, the IOP measured 6 hr after application of the working 1,7-phenanthroline solution dropped to 25.66, as shown in FIG. 6A.

### EXAMPLE 8

### Using 4,7-dihydroxy-1,10-phenanthroline to Lower IOP in the Morrison Model of Glaucoma

A solution of 4,7-dihydroxy-1,10-phenanthroline was prepared by dissolving 7 mg of 4,7-dihydroxy-1,10-phenanthroline in 22 μl methanol, 30 μl 1 N NaOH, and 626 μl of saline (0.9% NaCl) forming a 4,7-dihydroxy-1,10-phenanthroline solution having a pH of about 10.42. Using 1N HCl, the pH of the 4,7-dihydroxy-1,10-phenanthroline solution was adjusted to pH 7.7, which resulted in some precipitation of the drug. The 4,7-dihydroxy-1,10-phenanthroline solution was centrifuged at 10,000×g for about 5 min to remove the precipitate. The clear supernatant was removed and used for testing the Morrison model eye. The average intraocular pressure (IOP) in the eye with elevated pressure was 27.77 (average of nine readings) prior to application of the drug. IOP measured 6 hr after application of the 4,7-dihydroxy-1,10-phenanthroline solution dropped to 26.22, as shown in FIG. 6B.

### EXAMPLE 9

### Using 1,10-phenanthroline 5,6-dione to Lower IOP in the Morrison Model of Glaucoma

A solution of 1,10-phenanthroline 5,6-dione was prepared by dissolving 7 mg of 1,10-phenanthroline 5,6-dione in 44 μl methanol, and 88 μl dimethylsulfoxide (DMSO). A precipitate formed in the resultant solution (i.e. the entire drug was not solubilized). The 1,10-phenanthroline 5,6-dione solution with precipitate was centrifuged at 10,000×g for 5 min, and the clear supernatant was removed. 6 μl of the supernatant plus 97 μl of saline formed the working solution used in the Morison model of glaucoma. The average intraocular pressure (IOP) in eye with elevated pressure was 27.66 (average of nine readings) prior to application of the drug. IOP measured 6 hr after application of the drug dropped to 25.44, as shown in FIG. 6C.

### EXAMPLE 10

### Using Phenanthroline Derivatives to Treat Other Diseases of the Eye

It should be apparent to one of ordinary skill in the art that many derivatives of phenanthroline can also be used in formulations for treatment of other ocular diseases. Thus, another aspect of the current invention involves a method for treating an ocular disease or damage thereof in an animal, comprising administering to the animal, a composition containing an effective amount of an phenanthroline or derivative thereof in a pharmaceutically acceptable vehicle. The ocular diseases or damage contemplated by the inventors are selected from the group consisting of: uveitis, dry eye, diabetic retinopathy, and macular degeneration.

While the compositions and methods of this invention have described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents that are both chemically and physiologically related might be substituted for the agents described herein while the same or similar results would be achieved. All such similar substitutes and modifications to those skilled in the art are deemed to be within the spirit, scope and concept of the invention as defined by the appended claims.

### REFERENCES CITED

The following U.S. Patent documents and publications are incorporated by reference herein.

### U.S. Patent Documents

U.S. Pat. No. 3,809,714 issued to Hussain on May 7, 1974, titled “Novel Ester of [Methylamino)Methyl]Benzyl Alcohol.”
U.S. Pat. No. 3,839,584 issued to Hussain on Oct. 1, 1974, titled “Pharmaceutical Compositions Containing a Novel Ester of [Methylamino)Methyl]Benzyl Alcohol and Method of Using Same.”
U.S. Pat. No. 4,517,199 issued to York, Jr. et al., on May 14, 1985, titled “Method for Lowering Intraocular Pressure using Phenylimino-imidazoles.”
U.S. Pat. No. 4,911,920 issued to Jani, et al., on Mar. 27, 1990, titled “Sustained Release, Comfort Formulation for Glaucoma Therapy.”
U.S. Pat. No. 5,212,196 issued to House, et al., on May 18, 1993 titled, “Control of Post-Surgical Intraocular Pressure Using Clonidine Derivatives.”
U.S. Pat. No. 5,260,059 issued to Acott, et al., on Nov. 9, 1993, titled “Treatment of Open-angle Glaucoma by Modulation Matrix Metalloproteinases and Their Inhibitor.”
U.S. Pat. No. 5,688,819 issued to Woodward, et al., on Nov. 18, 1997, titled “Cyclopentane Heptanoic Acid, 2-cycloalkyl or Arylalkyl Derivatives as Therapeutic Agents.”
U.S. Pat. No. 6,224,848 issued to Mills on May 1, 2001, titled “Pharmaceuticals Providing Diagnosis and Selective Tissue Necrosis using Mossbauer Absorber Atom.”

### OTHER REFERENCES

CLARK A F, Yorio T. “Ophthalmic drug discovery.” Nat Rev Drug Discov. 2003
DAN J A, Honavar S G, Belyea D A, Mandal A K, Garudadri C, Levy B, Ramakrishnan R, Krishnadas R, Lieberman M F, Stamper R L, Yaron A. “Enzymatic sclerostomy: pilot human study.” Arch Ophthalmol. 2002 May; 120(5):548-53.
HEAD K. “Natural Therapies for Ocular Disorders Part Two: Cataracts and Glaucoma.” Altern Med Rev 2001; 6(2): 141-166.
MAMIYA K, Ohguro H, Ohguro I, Metoki T, Ishikawa F, Yamazaki H, Takano Y, Ito T, Nakazawa M. “Effects of matrix metalloproteinase-3 gene transfer by electroporation in glaucoma filter surgery.” Exp Eye Res. 2004 September; 79(3):405-10
NEUFELD A H. “Pharmacologic neuroprotection with an inhibitor of nitric oxide synthase for the treatment of glaucoma.” Brain Res Bull. 2004 Feb. 15; 62(6):455-9.
KAUSHIK S, Pandav S S, Ram J. “Neuroprotection in Glaucoma.” J Postgrad Med. 2003 January-March; 49(1):90-5.
SCHLOTZER-SCHREHARDT U, Lommatzsch J, Kuchle M, Konstas A G, Naumann G O. “Matrix Metalloproteinases and Their Inhibitors in Aqueous Humor of Patients with Pseudoexfoliation Syndrome/glaucoma and Primary Open-Angle Glaucoma.” Invest Ophthalmol Vis Sci. 2003 March; 44(3):1117-25.
WATSON P G, Young R D. “Scleral Structure, Organisation and Disease. A Review.” Exp Eye Res. 2004 March; 78(3):609-23.
WIEDERHOLD M., Thieme H., and Stumpff F. “The Regulation of Trabecular Meshwork and Ciliary Muscle Contractility.” Progress in Retinal and Eye Research. 2000 Vol. 19, No. 3, pp 271-295.
WOODWARD D F, Gil D W. “The inflow and outflow of anti-glaucoma drugs.” Trends Pharmacol Sci. 2004 May; 25(5):238-41.

What is claimed is:

1) A method of lowering intraocular pressure in an affected eye, comprising applying to the affected eye a pharmaceutically effective amount of at least one phenanthroline derivative in a suitable carrier.

2) The method of claim 1, wherein the phenanthroline derivative has a general structure of Formula I: 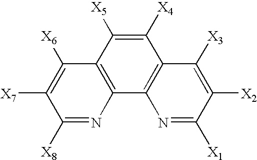
wherein X₁-X₈ are the same or different and comprise H, OH, O, CₙH₂ₙ₊₁ (wherein n=1-4), phenyl or substituted phenyl, Cl, —NO₂, or —CN.

3) The method of claim 2, wherein the phenanthroline derivative is 1,10-phenanthroline.

4) The method of claim 2, wherein the phenanthroline derivative is: 1,10-phenanthroline-5-acetonitrile; 2,9,dimethyl-1-10-phenanthroline; 3,4,7,8-tetramethyl-1-10-phenanthroline; 4,7,-dihydroxy-1-10-phenanthroline; 4,7,-dimethyl-1,10-phenanthroline; 4,7-diphenyl-1,10-phenanthroline; 4-methyl-1,10-phenanthroline; 5,6-dimethyl-1,10-phenanthroline; 5,6-dimethyl-1,10-phenanthroline; 5-chloro-1,10-phenanthroline; 5-methyl-1,10-phenanthroline; 5-nitro-1,10-phenanthroline; or their pharmaceutically acceptable analogues and derivatives.

5) The method of claim 1, wherein the phenanthroline derivative has a general structure of Formula II: 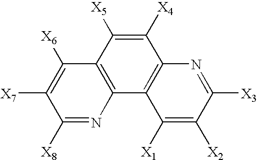
wherein X₁-X₈ are the same or different and comprise H, OH, O, CₙH₂ₙ₊₁ (wherein n=1-4), phenyl or substituted phenyl, Cl, —NO₂, or —CN.

6) The method of claim 5, wherein the phenanthroline derivative is 1,7-phenanthroline.

7) The method of claim 1, wherein the phenanthroline derivative has a general structure of Formula III: 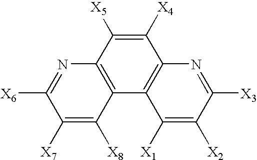
wherein X₁-X₈ are the same or different and comprise H, OH, O, CₙH₂ₙ₊₁ (wherein n=1-4), phenyl or substituted phenyl, Cl, —NO₂, or —CN.

8) The method of claim 7, wherein the phenanthroline derivative is 4,7-phenanthroline.

9) The method of claim 1, wherein a final composition concentration of the phenanthroline derivative is between about 0.05% and about 1.5% wt/volume of the final composition.

10) The method of claim 1, wherein the suitable carrier is: anionic, mucomimetic polymer; gelling polysaccharide; finely-divided drug carrier substrate; mineral oil; liquid petrolatum; white petrolatum; propylene glycol; polyoxyethylene; polyoxypropylene compound; emulsifying wax and water; or a combination thereof.

11) The method of claim 1, further comprising applying to the affected eye a pharmaceutically effective amount of at least a second compound;
wherein the second compound is a β-blocker, a prostaglandin, an α₂-agonist, or a miotic; and
the second compound is in a second suitable carrier from the phenanthroline derivative; or the phenanthroline derivative is mixed together with the second compound in the suitable carrier.

12) The method of claim 11, wherein the second compound is: pilocarpine, epinephrine, dipivefrin, levobunolol, timolol, betaxolol, carteolol, timolol, brimonidine, apraclonidine, dorzolamide, bimatoprost, unoprostone, travoprost, latanoprost, dichlorphenamide, acetazolamide, or methazolamide.

13) An ophthalmic composition for the treatment of glaucoma, comprising: a pharmaceutically effective amount of at least a first phenanthroline derivative and a second composition in a suitable carrier;
wherein the second compound is a β-blocker, a prostaglandin, an α₂-agonist, or a miotic.

14) The composition of claim 13, wherein the phenanthroline derivative has a general structure of Formula I: 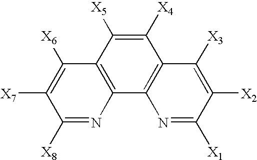
wherein X₁-X₈ are the same or different and comprise H, OH, O, CₙH₂ₙ₊₁ (wherein n=1-4), phenyl or substituted phenyl, Cl, —NO₂, or —CN.

15) The composition of claim 14, wherein the phenanthroline derivative is 1,10-phenanthroline.

16) The composition of claim 14, wherein the phenanthroline derivative is: 1,10-phenanthroline-5-acetonitrile; 2,9,dimethyl-1-10-phenanthroline; 3,4,7,8-tetramethyl-1-10-phenanthroline; 4,7,-dihydroxy-1-10-phenanthroline; 4,7,-dimethyl-1,10-phenanthroline; 4,7-diphenyl-1,10-phenanthroline; 4-methyl-1,10-phenanthroline; 5,6-dimethyl-1,10-phenanthroline; 5,6-dimethyl-1,10-phenanthroline; 5-chloro-1,10-phenanthroline; 5-methyl-1,10-phenanthroline; 5-nitro-1,10-phenanthroline; or their pharmaceutically acceptable analogues and derivatives.

17) The composition of claim 13, wherein the phenanthroline derivative has a general structure of Formula II: 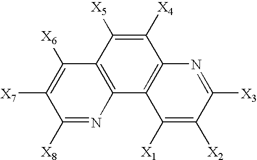
wherein X₁-X₈ are the same or different and comprise H, OH, O, CₙH₂ₙ₊₁ (wherein n=1-4), phenyl or substituted phenyl, Cl, —NO₂, or —CN.

18) The composition of claim 17, wherein the phenanthroline derivative is 1,7-phenanthroline.

19) The composition of claim 13, wherein the phenanthroline derivative has a general structure of Formula III: 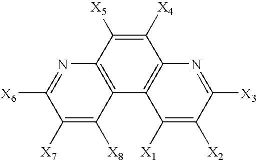
wherein X₁-X₈ are the same or different and comprise H, OH, O, CₙH₂ₙ₊₁ (wherein n=1-4), phenyl or substituted phenyl, Cl, —NO₂, or —CN.

20) The composition of claim 19, wherein the phenanthroline derivative is 4,7-phenanthroline.

21) The composition of claim 13, wherein the final composition concentration of the phenanthroline derivative is between about 0.05 and about 1.5 wt % of the final composition.

22) The composition of claim 13, wherein the suitable carrier comprises: anionic, mucomimetic polymer; gelling polysaccharide; finely-divided drug carrier substrate; mineral oil; liquid petrolatum; white petrolatum; propylene glycol; polyoxyethylene; polyoxypropylene compound; emulsifying wax and water; or a combination thereof.

23) The composition of claim 13, wherein the second compound is: pilocarpine, epinephrine, dipivefrin, levobunolol, timolol, betaxolol, carteolol, timolol, brimonidine, apraclonidine, dorzolamide, bimatoprost, unoprostone, travoprost, latanoprost, dichlorphenamide, acetazolamide, or methazolamide.

24) A method of treating glaucoma, comprising applying to an affected eye a pharmaceutically effective amount of a phenanthroline derivative having a final composition concentration of 1,10-phenanthroline in the range of about 0.05 and about 1.5 wt % in a suitable carrier, wherein the ophthalmic is anionic, mucomimetic polymer; gelling polysaccharide; finely-divided drug carrier substrate; mineral oil; liquid petrolatum; white petrolatum; propylene glycol; polyoxyethylene; polyoxypropylene compound; emulsifying wax and water; or a combination thereof.

25) The method of claim 24, further comprising applying to the affected eye a pharmaceutically effective amount of at least a second compound;
wherein the second compound is a pilocarpine, epinephrine, dipivefrin, levobunolol, timolol, betaxolol, carteolol, timolol, brimonidine, apraclonidine, dorzolamide, bimatoprost, unoprostone, travoprost, latanoprost, dichlorphenamide, acetazolamide, or methazolamide; and
the second compound is in a second suitable carrier from the 1,10-phenanthroline; or the 1,10-phenanthroline is mixed together with the second compound in the suitable carrier.
